(19) United States
(12) Patent Application Publication
(10) Pub. No.: US 2021/0007900 A1
(43) Pub. Date: Jan. 14, 2021

(54) Support X

(71) Applicant: Sonia Tamayo Warriner, Ponte Vedra Beach, FL (US)

(72) Inventor: Sonia Tamayo Warriner, Ponte Vedra Beach, FL (US)

(21) Appl. No.: 16/501,965

(22) Filed: Jul. 12, 2019

(51) Int. Cl. A61F 13/08; A41D 17/02

(52) U.S. Cl. CPC A61F 13/085; A41D 2300/32; A41D 2400/32; A41D 17/02

(57) ABSTRACT

This is a device that will make it easy for seniors and for the general public to use compression stocking to deal with medical problems of the lower legs. It will be produced in several sizes to fit men and woman and the various leg configurations.

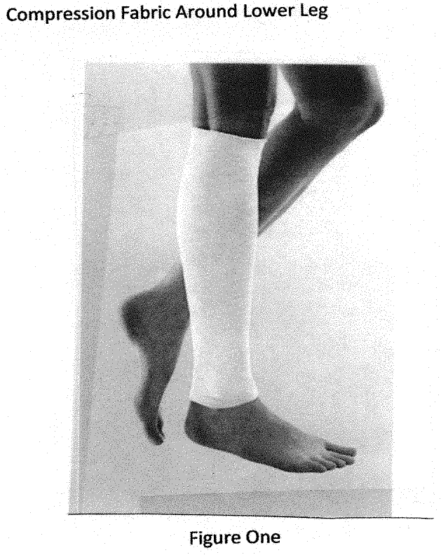

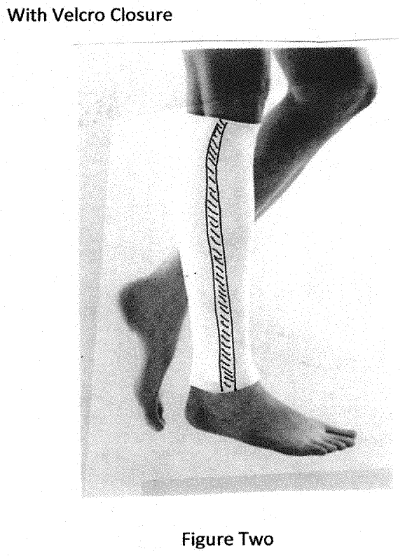

The purpose of this product is to create a Support Hose that will be easy to use and simple to wear.

The support hose will be made of very strong, stretch fabric that will wrap around the leg from the ankle to just under the knee. It will close with a line of Velcro that will run all the way up the leg.

The hose will be offered in several colors such as white, skin tone and black. It will also be produced in several designs and sizes to fit most female and male customers.

It is expected that this new type of support hose will have a great approval among adults with leg problems, particularly with the senior population that find the current type very difficult to wear.

1. A device that will be made of compression fabric in various colors and designs that will wrap around the lower leg and close with a line of Velcro for the purpose of providing support to improve circulation and reduce swelling.
